(19) United States
(12) Patent Application Publication
(10) Pub. No.: US 2020/0397720 A1
(43) Pub. Date: Dec. 24, 2020

(54) METHOD AND FORMULATION FOR TREATMENT OF WOUNDS BY NEW TISSUE REGENERATION WITH REDUCED TRAUMA AND SCAR TISSUE FORMATION

(71) Applicant: Charles Huntington Gallina, Cordova, IN (US)

(72) Inventor: Charles Huntington Gallina, Cordova, IN (US)

(21) Appl. No.: 16/449,431

(22) Filed: Jun. 23, 2019

(51) Int. Cl. A61K 31/07; A61K 31/573; A61K 9/00; A61P 17/02

(52) U.S. Cl. CPC A61K 31/07; A61K 31/573; A61K 45/06; A61P 17/02; A61K 9/0014

(57) ABSTRACT

The present teachings are directed to a method of promoting new tissue regeneration and wound healing in a mammal by inhibiting production of at least one of inflammatory cells, neutrophils, macrophages, and leukocyte lineages in a wound area, limiting inflammation of the wound area, and decreasing degradation of platelets, thereby modulating the coagulation process and formation of a clot by application of a formulation containing a steroid, a mixture of at least two antibiotics, and a vitamin A derivative. The method further includes applying the formation to a wound thereby reducing bleeding from any opened blood vessels in the wound by vasoconstriction, and preventing formation of a clot within the wound. The disclosed healing method reduces the need for stitches or sutures, minimizes scarring, and reduces patient trauma by quickly reducing bleeding, pain and inflammation. Also disclosed is a healing formulation composed of a steroid, a mixture of at least two antibiotics, and a vitamin A derivative.

### BACKGROUND

### Field of the Invention

The present teachings relate to a method for healing wounds and a formulation for healing wounds both of which heal wounds through a healing process different from an untreated wound.

### Discussion of the Related Art

Basic research about the wound healing process describes a three or four phase process (some researchers combine hemostasis and inflammation into one phase) involving the hemostasis, inflammation, proliferation, and remodeling (also known as maturation) phases. The process encompasses overlapping biochemical and cellular stages. The healing process is described in more detail below.

Inflammation Phase: The inflammatory phase is typically the longest, and is consider the most critical as its outcome can affect the other stages. In many cases, these healing processes can mutate and become chronic and non-curable which in severe cases can lead to gangrene, amputation and sepsis.

A graphical overview of the inflammatory phase as it occurs naturally is depicted in FIG. 12 from Singer, A. J. and Clark, R. A. F. (1999) Cutaneous wound healing. The New England Journal of Medicine, 341, 738-746. http://dx.doi.org/10.1056/NEJM199909023411006. Various factors involved in the inflammatory phase are FGF, basic fibroblast growth factor; IGF, insulin-like growth factor; KGF, keratinocyte growth factor; PDGF, platelet-derived growth factor; TGF, transforming growth factor; VEGF, vascular endothelial growth factor.

The first step in the phase is clot formation or hemostasis. This five to ten minute phase begins with vasoconstriction at the very beginning of the wound healing process. Some researchers consider hemostasis as the first part of the overall inflammation phase.

Immediately after tissue injury there is a vascular hemorrhage, which in addition to rupturing cell membranes, releases inflammatory factors into the surrounding extracellular matrix (“ECM.”) This release begins a chemical signaling to alert the host of cell death. Following this tissue injury, there is a period of vasoconstriction mediated by epinephrine, norepinephrine, prostaglandins, serotonin and thromboxane. This natural vasoconstriction period causes temporary blanching of the wound and functions to reduce hemorrhage immediately following tissue injury, aid in platelet aggregation, and keep healing factors within the wound. This natural vasoconstriction usually lasts five to ten minutes.

There is also mechanical trauma to these capillary blood vessels that can cause these capillaries to immediately spasm and constrict. However, there is usually no pain during this first five to ten minute time period, and thus, with small wounds many people do not realize they have been injured until they notice the bleeding. However, if there is more trauma to the wound area and bleeding restarts, the healing process starts over but with pain sensations. This additional trauma also has the potential to infection of the wound area to occur.

The subendothelial collagen of the damaged capillary walls is exposed and the circulating platelets attach to the exposed collagen surfaces through adhesive glycoproteins. The aggregation of platelets results in the formation of the primary platelet plug. Aggregation of the platelets enables them to release growth factors such as PDGF (“platelet-derived growth factor,”) TGF (“transforming growth factor”)-β1, TGF-β2, TGF-β3, TGF-a, serotonin, histamine, and bradykinin. These growth factors start the coagulation pathway by activating cleavage of prothombin to form thrombin which in turn converts soluble fibrinogen into insoluble strands of fibrin. After the platelets attach to the exposed endothelial tissue around the wound opening, the fibrin, a sticky component, covers these platelets binding them together with the tissue, and forming the primary platelet plug. This primary platelet plug seals the wound opening and stops the bleeding. The platelet plug (or clot) also traps and acts as a nest for the inflammatory cells, such as neutrophils, macrophages M1, TGF-α, TGF-β1, TGF-β2, TGF-β3, other leukocytes, plasma proteins, cytokines and growth factors. These inflammatory cells will continue to secrete cytokines and growth factors that start and direct the repertory cascade of biological processes. This nest can be seen in FIG. 22 at the bottom of the open wound area.

Cytokines have been considered to be “messengers” within the cell that transmit signals of pain from one area of the body to the brain. Additionally, cytokines can trigger various responses within the body in reaction to events such as cuts to the dermis.

It is conventional understanding that healing can only start once the clot is lysed because the clot prevents both angiogenesis and wound contraction from occurring. Additionally, the clot is located where the granulation tissue would be deposited, and without granulation tissue angiogenesis cannot take place.

The cells trapped in the platelet plug repetitively release pro-inflammatory factors causing the release, or flow, of histamine from the platelets and local mast cells, and activating what is known as the Hageman factor (or “Factor XII.”) The Hageman factor then starts what is known as the Kinin or contact activation pathway. This pathway eventually produces the peptide, bradykinin, which is a very potent endothelium-dependent vasodilator, and is involved in the pain mechanism through its binding to neural receptors.

Both bradykinin and histamine are released into the blood plasma from the surrounding mast cells and stimulate vasodilation. The vasodilation greatly increases vascular permeability of the local capillary blood vessel walls which allows for the continuous flow of blood plasma containing platelets, leukocytes, and other inflammatory factors from the engorged capillaries into the wound area. The introduction of more inflammatory factors into the wound area can cause edema, pain, and tissue damage along with increased cytokine signaling.

The increased capillary permeability and resulting decreased blood velocity facilitates the migration of the various leukocytes. Generally first to arrive are neutrophils that clean up and sterilize the wound area. Neutrophils are very toxic and can cause cell damage but are short lived, with life-spans of about 48 hours. The next arriving inflammatory cells are the monocytes. They mature into M1 and M2 macrophages, which in conjunction with resident tissue macrophages orchestrate the wound healing process.

The M2 macrophages engulf cells of damaged tissue and bacterial debris, while M1 macrophages secrete multiple growth factors to activate local endothelial cells, fibroblasts, and keratinocytes to begin their respective repair functions. More than twenty different cytokines and growth factors are known to be secreted by these macrophages. This stage concludes the inflammatory phase.

Proliferation Phase: The second phase of the wound healing process involves fibroplasias when fibroblasts divide and produce fibronectin and collagen tissue. Granulation tissue is also formed by angiogenesis where the endothelial cells of the capillary blood vessels sprout buds and develop new capillary networks within the newly formed collagen. The new collagen is characterized by its beefy-red appearance. This feature appears in second and third intention wounds primarily.

Contraction of open wounds is a process by which surrounding tissue is pulled circumferentially toward the wound. Wound contraction decreases the size of the wound and thereby decreases the amount of new tissue formation required to heal. This contraction leads to a smaller overall wound, and thus a faster healing process.

Contraction allows the wound to close and heal much more rapidly than just epithelialization which can start immediately after a tissue injury. Epithelialization after an injury can cause morphological changes in the keratinocytes in the wound area. During epithelialization, keratinocytes can line up along the edges of the wound, while the epidermis thickens and keratinocyte daughter cells flatten and migrate into the wound matrix. The migration of the keratinocytes across the wound bed will continue until migrating cells meet one another, usually in the middle of the wound bed, and contact inhibition will stop the migration.

It has been observed that a lengthy contraction phase can lead to scarring and loss of function. Thus, there is great interest in ways to shorten the contraction phase.

Maturation and Remodeling Phase: The final phase of the wound healing process is also known as the scar maturation phase. Scar tissue is a product of collagen and fibrin deposition by fibroblasts during the proliferation stage.

Some known ointments contain zinc oxide as a promotor of blood coagulation in order to, as presently understood, promote healing of cuts and burns. See, for example, U.S. Pat. No. 6,375,942 B1. The presently disclosed formulation, as set forth herein, does not promote blood coagulation, in fact, as set forth below, it heals from inside the wound and outward, without formation of a clot in the wound area.

It would be desirable to be able to control the wound healing process, particularly the inflammatory process, to decrease the chances of mutation and undesirable outcomes, like formation of chronic wounds or ulcers. Control of the wound healing process can greatly improve a patient's quality of life.

There is a need for a method and formulation that achieves healing of a wound while minimizing the pain, risk of infection, and scarring that typically occurs in both an untreated wound and wounds as treated with known methods.

### SUMMARY OF THE PRESENT DISCLOSURE

The present teachings are directed to a topical formulation for healing wounds that heals wounds through a different process than that which occurs in either an untreated wound or a wound as treated with known methods. The presently disclosed formulation and method result in wounds healing without a blood clot or scab formation and with greatly reduced pain and/or inflammation.

The present disclosure is directed to a method of promoting new tissue regeneration in a mammal by providing a formulation comprised of a steroid, a mixture of at least two antibiotics, and a vitamin A derivative, applying the formation to a wound to thereby reduce bleeding from any opened blood vessels in the wound by vasoconstriction, and preventing formation of a clot within the wound.

The present disclosure also includes a formulation for wound healing containing at least one anti-inflammatory steroid, at least one antibiotic, and a vitamin A derivative.

A method of wound healing, also taught by the present disclosure, includes inhibiting production of at least one of inflammatory cells, neutrophils, macrophages, and leukocyte lineages in a wound area, limiting inflammation of the wound area, and decreasing degradation of platelets, and thereby modulating the coagulation process and formation of a clot by application of a formulation comprised of a steroid, a mixture of at least two antibiotics, and a vitamin A derivative.

The present teachings also include a method of preparing a pharmaceutical formulation by providing a pharmaceutical base formulation, providing pharmaceutical grade 1% hydrocortisone, mixing the base formulation with the hydrocortisone to produce a first mixture, aging the first mixture at room temperature for a first time period, providing at least three different antibiotic-containing formulations, mixing the at least three antibiotic-containing formulations together to produce a second mixture, aging the second mixture at room temperature for a second time period, then adding the second mixture to the first mixture to produce a third mixture, aging the third mixture at room temperature for a third time period, providing a vitamin A derivative-containing formulation, adding the vitamin A derivative-containing formulation to the third mixture to produce a final formulation, and aging the final formulation at room temperature for a fourth time period.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 is a photograph of a female hip replacement patient;

FIGS. 2A and 2B are photographs of a female hip replacement patient;

FIG. 3 is a photograph of a male head wound patient;

FIG. 4 is a photograph of a male head wound patient;

FIG. 5 is a photograph of a burn wound to a hand;

FIG. 6 is a photograph of a healed burn wound to a hand;

FIG. 7 is a photograph of a cut forearm;

FIG. 8 is a photograph of a cut forearm;

FIG. 9 is a photograph of a cut forearm;

FIG. 10 is a photograph of a cut forearm;

FIG. 11 is a photograph of a cut forearm;

FIG. 12 is a graphical representation of the inflammatory phase;

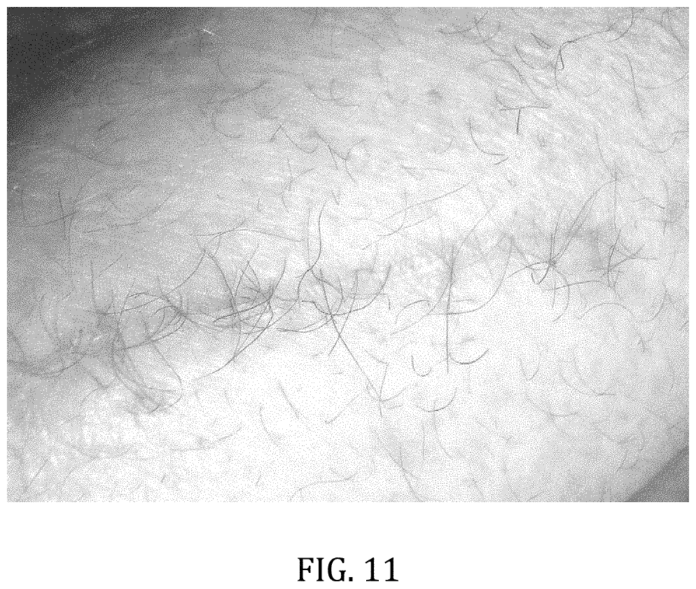

FIG. 13 is a photograph of an infected foot;

FIG. 14 is a photograph of an infected foot;

FIG. 15 is a photograph of an infected foot;

FIG. 16 is a photograph of an infected foot;

FIG. 17 is a graphical representation of skin wound;

FIG. 18 is a graphical representation of skin wound;

FIG. 19 is a photograph of a cut thumb;

FIG. 20 is a photograph of a cut thumb;

FIG. 21 is a photograph of a thumb, and

FIG. 22 is a graphical representation of a wound reaction.

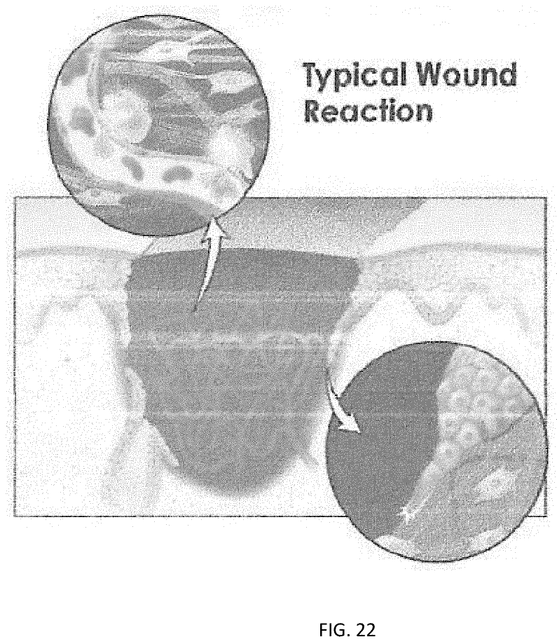

### DETAILED DESCRIPTION

The present understanding of the natural wound healing process in a human body is that a three phase process involving the inflammation, proliferation, and remodeling (also known as maturation) phases occurs. Some researchers divide the inflammation phase into separate hemostasis and inflammation phases. The overall process encompasses overlapping biochemical and cellular stages.

The Presently Taught Healing Process:

The presently disclosed method causes the capillary blood vessels in the wound area to constrict, making their walls thicker by the contraction of their cells making the capillary walls impermeable. This vasoconstriction lasts throughout the entire disclosed wound healing method.

The permanent vasoconstriction of the damaged capillary walls makes their walls impermeable, and does not allow blood plasma that carries leukocytes, platelets and other pro-inflammatory cells into the tissue surrounding the wound site. By not coming into contact with the cells local to the wound area, the leukocytes, platelets and other pro-inflammatory cells do not excite them by means of cytokine signaling. Vasoconstriction also can be observed in cases where histamine is released into the blood plasma.

Damaged cells in the wound area can be lysed by other local cells and macrophages that are present in the wound area prior to the wound, and are not delivered there by the blood system. A simple small splinter in your finger is an example of this occurrence. In the case where a small splinter causes no bleeding or infection, it eventually will disappear into the skin. The splinter is lysed by locally available lysosomes. Because the capillary blood system did not become involved, there was no delivery of neutrophils or monocytes to the site to phagocytize the foreign splinter.

In the presently disclosed healing method, platelets cannot enter the wound area and any platelets that are found in the wound area can be neutralized by the antibiotics from the presently disclosed formulation. Additionally, the antibiotics can destroy any bacteria that entered into the wound at the time of trauma, and turn off their chemical signaling. The antibiotics can also be neutralizing antibiotics, and play a factor in the permanent vasoconstriction process and in turning off other cell actions. Another function of the antibiotics is to eradicate any naturally occurring staph and strep bacteria.

In the presently disclosed healing method, the vitamin A derivative can stimulate endothelial cells to sprout new buds that become the new capillaries in the wound area inside new granulation tissue. The vitamin A derivative can also stimulate epithelial stem cells producing daughter cells to thereby increase the thickness of epithelial tissue present at the wound edges. This epithelial tissue can line up at the wound edges, and form new epithelial tissue which can stretch across and close the wound.

In the presently disclosed healing method, where there is no, or essentially little, formation of a clot as a result there are no, or essentially very few, trapped platelets or other proinflammatory cells in the wound area. These platelets typically signal the fibroblasts to produce scar forming collagen, however in the presently disclosed healing method, such scar forming collagen is not formed.

Without the formation of the clot the presently disclosed healing method allows for earlier formation and deposition of granulation tissue in the wound bed. Additionally, the absence of a clot, which typically physically holds a wound open, allows the wound to start contraction and closure earlier.

The presently disclosed wound healing formulation can prevent both vascular dilation and permeability by not allowing the flow of platelets and leukocytes and other inflammatory factors into the wound tissue. The presently disclosed wound healing formulation also does not allow the neutrophils and/or the pro-inflammatory M1 macrophages into the wound bed. By keeping these tissue damaging components out of the wound area, the presently disclosed wound healing formulation also can help prevent their subsequent inflammatory cytokine signaling process.

The presently disclosed wound healing formulation can change the healing process into a regenerative process by eliminating the lowest level of inflammatory cells and/or processes, such as, neutrophils, M1 macrophages, leukocyte lineages and microbial infections from the wound area. Additionally, by not allowing the degradation and subsequent signaling from platelets, and modulating certain lineages in the coagulation process, a robust inflammatory response does not occur.

Healing of wounds along with generation of new tissue involves coordinated cell activation, cell division, chemotaxis, migration and differentiation of many cell types. These actions are believed to be controlled by both growth factors and certain cytokines. All phases of the wound healing process are believed to be directly or indirectly regulated by cytokines. It is believed that the regulation of these cytokines and other mediators will control cell actions and not just the absence or presence of one or more cytokines.

Certain cytokines appear to have more than just one specific effect on cells. It is believed that the presently disclosed wound healing formulation modulates the regulation of these cytokines and other mediators through several different mechanisms including, for example: a) interacting with specific DNA sequences for activation or suppression of genes responsible for cytokine production, b) protein to protein interaction inhibiting the activities of various pro-inflammatory transcriptional factors, and c) enhancing the degradation of specific mRNA's.

In the presently disclosed healing method, certain coagulation lineages can be controlled, and thus significantly prothrobin is prevented from being converted into thrombin which in turn prevents conversion of fibrinogen into fibrin, and formation of the primary blood clot does not occur.

The primary blood clot is believed to interfere with contraction and closure of the wound. Additionally, the clot also serves as a nest or reservoir for the soluble inflammatory signaling proteins such as, for example, neutrophils, M1, TGF-α, TGF-β1, TGF-β2, TGF-β3, PDGF, VEGF, and FGF. The presence of the clot allows for these inflammatory signaling proteins to keep the inflammatory process active for a longer time whereas removing the clot limits the repetitive cytokine and growth factor signaling to a fewer protein groups.

The presently disclosed formulation for wound healing can prohibit the formation of the primary blood clot to form in the wound. Instead, bleeding is stopped by vasoconstriction of the blood vessels through control of bradykinin peptide and histamine. The vasoconstriction is accomplished quicker than formation of a clot. It is presently understood that the vasoconstriction does not allow for the blood vessels to become permeable and thereby halts the immigration of more leukocytes to the wound bed. Furthermore, the vasoconstriction does not allow more platelets to enter the wound site.

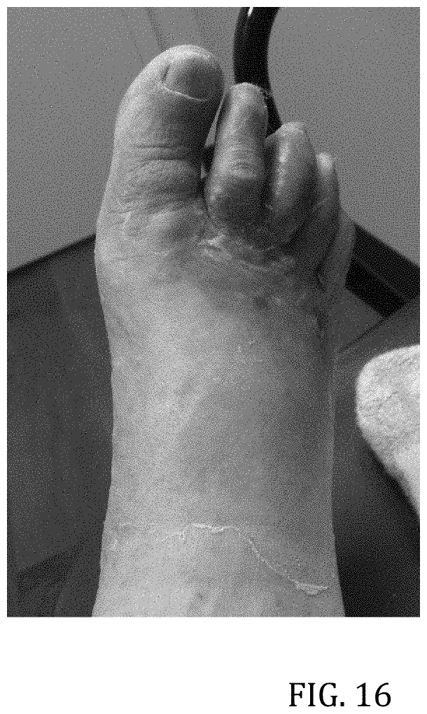

FIG. 17 illustrates that the application of the presently disclosed wound healing composition prevents formation of the clot in the wound site and entry of the various factors into the wound site.

It is the present understanding that the disclosed formulation limits blood loss, vasoactive mediators, and tissue factor from building up in the wound site, and thereby limits or modulates platelet activation. This effect of the disclosed formulation effectively puts and maintains the wound site in hemostasis. With both hemostasis and vasoconstriction of the damaged capillary blood vessels going on, the normal influx of bacteria to the blood supply is greatly reduced or eliminated.

With the wound site in hemostasis, the oxygen level drops and activates the existing M2 tissue macrophages, and within hours these macrophages start to phagocytize the debris and dead tissue in the wound site. These macrophages proliferate fibroblasts and secrete factors that attract and stimulate endothelial cells and their proliferation. This proliferation is believed to promote angiogenesis and formation of both new tissue and granulation tissue.

In the typical wound healing process, neutrophils are generally the first cells to arrive at the wound site, however they generally believed to be unessential to wound healing. Additionally, neutrophils are known to be toxic and destructive so the presently disclosed formulation keeps them out of the wound site. M1 macrophages found in the blood are also eliminated, or at least reduced, by vasoconstriction.

The currently disclosed method of wound healing avoids blood coagulation (or clot formation) and formation of a scab in the wound interior though suppression of the production of cytokines. The decrease of cytokine secretion is believed to be achieved by the reduction in NF-κB (“Nuclear Factor Kappa B”) supplied to the cell's nucleus. As set forth above, it is generally accepted that the wound healing process begins with formation of a blood clot and the subsequent scar formation, in stark contrast, the currently disclosed method takes a different approach by reducing the production of certain key cytokines.

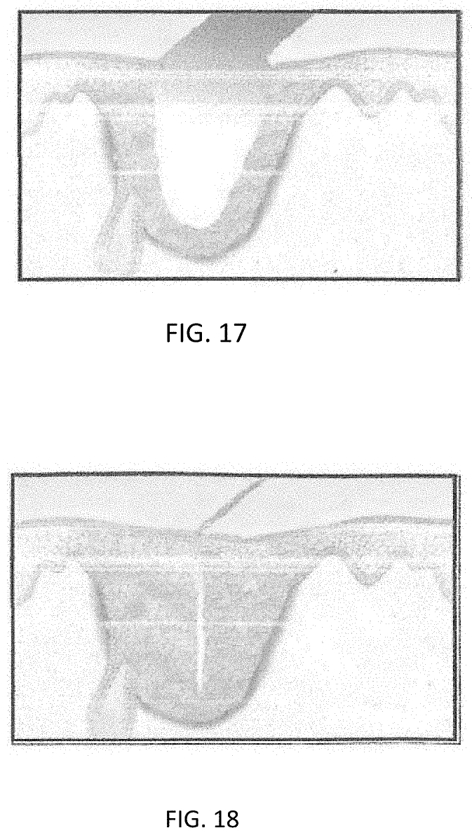

FIG. 18 illustrates the effect of the presently disclosed wound healing composition on allowing the connecting tissue from the sides of the wound site to contact and effectuate closure of the wound without or greatly reduced scar tissue formation.

By limiting the amount of fibrin collection in the wound area, the present formulation is understood to prevent the formation of the clot in the wound area. The ability of the present formulation to inhibit fibrin collection may also be utilized in the treatment of other conditions or diseases caused by excessive fibrin generation, or build-up of fibrin in the body.

The present formulation can comprise a 1% hydrocortisone cream comprised of hydrocortisone in a base containing glyceryl monostearate, polyoxyl 40 stearate, glycerin, paraffin, stearyl alcohol, isopropyl palmitate, sorbitan mono-stearate, benzyl alcohol, potassium sorbate, lactic acid, or purified water.

The presently disclosed formulation further comprises a mixture of at least three antibiotics that are mixed together and allowed to age in sterile conditions for at least two weeks and up to four weeks. Three weeks of aging at room temperature appear to provide the best results.

One possible combination of three antibiotics is neomycin sulfate, polymyxin B sulfate, and bacitracin.

Neomycin sulfate can be utilized in an initial concentration of 3.5 mg per gram in a white petroleum base, or 600 μg of neomycin per mg. It is the sulfate salt of neomycin B&C, and is produced by the growth of Streptomycin fradiae.

Polymyxin B sulfate can be utilized at an initial concentration of 6,000 polymyxin units in a white petroleum base. It is the sulfate salt of polymyxins B1 and B2 which are produced by the growth of Bacillus polymyxa.

Bacitracin can be utilized at an initial concentration of 500 bacitracin units in a white petroleum base. It is the salt of bacitracin, and is produced by the growth of the Licheniformis group of Bacillus subtilis organism.

While the three above-listed antibiotics are generally preferred in the presently disclosed formulation, the present disclosed formulation can utilize many other antibiotics as called for by the conditions in which the formulation is used. Conditions to be considered can include, for instance, patient sensitivities to formulation components, strength of antibiotic required, storage conditions, longevity of antibiotics or other formulation components, and interactions between components of the formulation.

Aminoglycoside are one general category of antibiotics (including streptomycin, kanamycin, tobramycin, gentamicin, and neomycin.)

Possible antibiotics that can be included in the presently disclosed formulation include, without limitation, antibiotics selected from the group consisting of Neomycin Sulfate, Polymyxin B Sulfate, Polymoxin E, Polymoxin M, Bacitracin, Streptomycin, Kanamycin, Tobramycin, Tobrex, Gentamicin, Garamycin, Paromomycin, Neomycin, plus other Aminoglycoside antibiotics. Additionally, the Rifamycin family of antibiotics including Rifampicin, Rifaldazine, Rofact, Rfamycin, the Cephalosporins family of antibiotics, N, C, P and the Beta Lactamase Inhibitors, Ancef, Kefazol[cefazolin], Ceclor, Cefazolin Cefaclor, Cefdinir, Ceftin, Zinacef [Cefuroxime], Duricef, Velocef, Biocef, Panixine [cefadroxil], Keflex, Keftabs [cephalexin], Maxipime [cefepime], Rocephin [ceftria], Avibactam/Ceftazidime, Ceftolozane/Tazobactam [Teflaro], Vantin, Omnicef, Cedax, Claforan, Vantin, Spectracef, Tazicef, Fortaz, Ceptaz, Cefobid, Cefizox and Spectracef [SUPRAX]. Other possible antibiotics include Penicillins, Penicillin V [Phenoxymethylpenecillin], Penicillin G [BenzylPenicillin] [pfizerpen, permapen], Amoxcillin [amoxil], Ampicillin [unasyn] [Polycillin], Carbenecillin, Cloxacillin [Cloxapen], Augmentin [amox/clavulonate], Nafcillin [nallpen], Oxacillin [bactocill], Dicloxacillin [Diclocil], Flucloxacillin [Floxapen], and also Glycopeptides, anti-infective antibiotics, Vancomycin [for MRSA], Teicoplanin, Telavancin, Ramoplanin, Decaplanin, and the anti-tumor antibiotic, Bleomycin. Also possible are Ciprofloxacin, Nalidixic Acid, Norfloxacin, Levofloxacin, Gemifloxacin, and Moxifloxacin. The tetracyclines, which are known for exhibiting cytokine modulation including Doxycycline, Methacycline, Minocycline, Oxytetracycline, Chlortetracycline, and Lymecycline can be utilized in the presently disclosed formulation.

The sulfonamides are also possible antibiotics for the presently disclosed formulation including Co-Trinoxazole [septrin], Sulfadiazine, Sulfamethoxazole [gantanol], Trimethoprim-Sulfamethoxazole [bactrim, bactrimds, septra, septrads], Trimethoprim [trimpex, proloprim, and primsol], Sulfasalazine [azulfidine EN-tabs, Azulfidine, and Sulfazine], Sulfisoxazole [Gantrisin], Bactropin, Sulfamethoxazole, Sulfacetamide, Aczone, Cotrimoxazole, Sulfathiazole, and Silver Sulfathiazole.

Even more antibiotics for possible inclusion in the presently disclosed formulation include Clindamycin, Macrolides, Roxithromycin [Roximycin], Erythrocin, Zmax, Zithromax, Biaxin, Ery-Tab, Dificid, Erythrocin Stearate Filmtab, Biaxin XL, E.E.S. Granules, E.E.S.-400 Filmtab, Eryc, EryPed, Erythrocin Lactobionate, Ilosone, PCE Dispertab, Erythromycin[E-Mycin], Josamycin, [Josalid], Pristinamycin, [Pyostacine], Azithromycin, Clarithromycin, Spiramycin [Spirex], Telithromycin [Ketek], Dirithromycin [Dynabac], Fusidic Acid, Fucidin, Fucithalmic, Stafine, Sodium Fusidate, Fucicort, Fucibet, Fucizon, Fusimax, Foban, Stanicid, Fugen, Optifucin, Taksta, Phudicin, Usidin and the rest of its tradename family and preparations formulated, Amphenicols, such as, Chloramphenicol [Clorin], Thiamphenicol [Biothicol]; Nitroimidazoles, such as, Metronidazole [Flagyl]; Oxazolidinones, such as, Linezolid [Zyvox] and Tedizolid [Sivextro]; Pleuromutilins, such as, Retapamulin [Altabax]. Finally, antibiotics which can be used in the presently disclosed formulation include Nitrofurantoin, Trimethoprim, Mupirocin, Gramicidin, Fosfomycin, and Bacitracin.

Various embodiments of the presently disclosed formulation contain at least one steroid which may be selected from the group consisting of hydrocortisone, hydrocortisone valerate, hydrocortisone butyrate, desonide, amcinonide, betamethasone valerate, clobetasone butyrate, desoximetasone, diflucortolone valerate, fluocinolone acetonide, mometasone furoate, prednicarbate, triamcinolone acetonide, flurandrenolide, fluticasone propionate and also high potency steroids including betamethasone dipropionate, halcinonide, and triamcinolone acetonide, and even higher potency steroids including clobetasol propionate, halobetasol propionate, temovate, dexamethasone, diflucortolene, flumethasone, fluocinonide, ethylprednisolone, budesonide, alclometasone dipropionate, methylprednisone, and prednisone.

Various embodiments of the presently disclosed formulation contain at least one retinoid which may be selected from the group consisting of retinol, retinal, tretinoin or all-trans retinoic acid [“ATRA”], retinyl palmitate, isotretinoin, alitretinoin, etretinate and its metabolite acitretin, adapalene, bexarotene, and tazarotene.

The topically applied retinoids improve skin damage by modifying cellular differentiation programs, initiating the increase of epidermal proliferation leading to epidermal thickening, compaction of the stratum corneum, (the horny outer layer of the skin), and biosynthesis and deposition of glycosoaminoglycans [Griffiths et al. 1993].

ATRA's effect on lowering inflammatory cytokines, including IL-6 and TNF-α, was demonstrated in a study of high fat diet-induced atherosclerosis in rabbits. [Zhou et al., J. Biomedicine and Biotechnology, “All-trans-Retinoic Acid Ameliorated High Fat Diet-Induced Atherosclerosis in Rabbits by Inhibiting Platelet Activation and Inflammation,” Vol. 2012, Article ID 259693, 2012.]

The present disclosure is believed to be the first instance of the use of vitamin A derivatives, such as, tretinoin, for inside a wound for healing purposes. Heretofore, tretinoin has been used only as a topical application for dermatologic conditions.

Treating the inside of wounds with tretinoin results in the deposition of reticulin, or Type 3 collagen, fibers and generation of type 1 procollagen secreted into the dermal extracellular space. The type 1 procollagen is believed to then undergo enzymatic processing which arranges the procollagen into a triple helix configuration.

Further disclosed herein is a method of promoting tissue regeneration in a mammal by providing a formulation comprised of a steroid, a mixture of at least two antibiotics, and a vitamin A derivative, and applying the formation to a wound, thereby reducing bleeding from any opened blood vessels in the wound by vasoconstriction, and preventing formation of a clot within the wound.

It is presently understood that vascular constriction inhibits the introduction of both histamine and bradykinin from mast cells outside of the wound site. Histamine and bradykinin are needed in the normal coagulation process, and their absence causes the non-formation of a clot within the wound bed.

The presently disclosed method further includes reducing a flow of one or more members of the group consisting of leukocytes, peptide regulatory factors, glycoproteins, cytokines, membrane cytokines, soluble cytokines, growth factors, TGF-b, PDGF, platelets, TNF-a, IL8, chemokines, histamine, bradykinin, neutrophils, M1 macrophages, fibroblasts, keratinocytes, plasmin, prothrombin, prostaglandins, kinins, liposomal enzymes, and thrombin into the wound.

Tumor necrosis factor is a cytokine involved in systemic inflammation and is a member of a group of cytokines that all stimulate the acute phase reaction. IL-8 is a chemotactic factor that attracts neutrophils, basophils, and T-cells, but not monocytes. It is also involved in neutrophil activation. It is released from several cell types in response to an inflammatory stimulus.

The absence of one or more members of the group consisting of leukocytes, prostaglandins, kinins, histamine, and liposomal enzymes is understood to decrease vascular permeability and inflammation in the wound.

In some embodiments of the present method flow of histamine into the wound is reduced, and thereby the absence of histamine decreases vascular dilation in the wound area. Some embodiments of the presently disclosed method further include reducing the flow of one or more members of the group consisting of histamine, bradykinin, degranulating platelets, and chemokines to prevent the formation of a fibrin clot.

The present method by preventing formation of a fibrin clot undercuts the clot's role as a functioning reservoir for growth factors and cytokines. Additionally, the clot can no longer serve as a provisional matrix for cell migration including contaminating bacteria and other foreign particles and debris that would normally be removed by an oxidative burst phenomenon which utilizes destructive enzymes and can increase the potential for neutrophil-mediated tissue destruction.

It is further understood that the present method by causing the absence of histamine and bradykinin decreases both vascular permeability and dilation in the wound area, and reduces pain in the wound area for the entire duration of the healing process.

The presently disclosed method further comprises stopping the formation of one of more of prothrombin, thrombin, and prothrombinase complex. The conversion of fibrinogen to fibrin is also reduced by the presently disclosed method.

Further disclosed herein is a formulation for wound healing containing at least one anti-inflammatory steroid, at least one antibiotic, and a vitamin A derivative.

This currently disclosed formulation includes a steroid which comprises one or more steroid selected from the group consisting of hydrocortisone, hydrocortisone valerate, hydrocortisone butyrate, desonide, amcinonide, betamethasone valerate, clobetasone butyrate, desoximetasone, diflucortolone valerate, fluocinolone acetonide, mometasone furoate, prednicarbate, triamcinolone acetonide, flurandrenolide, fluticasone propionate, betamethasone dipropionate, halcinonide, triamcinolone acetonide, clobetasol propionate, halobetasol propionate, temovate, dexamethasone, diflucortolene, flumethasone, fluocinonide, ethylprednisolone, budesonide, alclometasone dipropionate, methylprednisone, and prednisone.

In some embodiments of the presently disclosed formulation, the mixture of at least two antibiotics comprises two or more antibiotics selected from the group consisting of neomycin sulfate, polymyxin B sulfate, polymyxin E, polymyxin M, bacitracin, streptomycin, kanamycin, tobramycin, gentamicin, paromomycin, Tobrex, Garamycin, Neomycin, Rifampicin, Rifaldazine, Rofact, Rfamycin, Cephalosporins N, C, P, Ancef, Kefazol[cefazolin], Ceclor, Cefazolin Cefaclor, Cefdinir, Ceftin, Zinacef [Cefuroxime], Duricef, Velocef, Biocef, Panixine [cefadroxil], Keflex, Keftabs [cephalexin], Maxipime [cefepime], Rocephin [ceftria], Avibactam/Ceftazidime, Ceftolozane/Tazobactam [Teflaro], Vantin, Omnicef, Cedax, Claforan, Vantin, Spectracef, Tazicef, Fortaz, Ceptaz, Cefobid, Cefizox, Spectracef [SUPRAX], Penicillins, Penicillin V [Phenoxymethylpenecillin], Penicillin G [BenzylPenicillin] [pfizerpen, permapen], Amoxcillin [amoxil], Ampicillin [unasyn] [Polycillin], Carbenecillin, Cloxacillin [Cloxapen], Augmentin [amox/clavulonate], Nafcillin [nallpen], Oxacillin [bactocill], Dicloxacillin [Diclocil], Flucloxacillin [Floxapen], Glycopeptides, anti-infective antibiotics, Vancomycin, [for MRSA], Teicoplanin, Telavancin, Ramoplanin, Decaplanin, Bleomycin, Ciprofloxacin, Nalidixic Acid, Norfloxacin, Levofloxacin, Gemifloxacin, Moxifloxacin, Doxycycline, Methacycline, Minocycline, Oxytetracycline, Chlortetracycline, Lymecycline, Co-Trinoxazole [septrin], Sulfadiazine, Sulfamethoxazole [gantanol], Trimethoprim-Sulfamethoxazole [bactrim, bactrimds, septra, septrads], Trimethoprim [trimpex, proloprim, and primsol], Sulfasalazine [azulfidine EN-tabs, Azulfidine, and Sulfazine], Sulfisoxazole [Gantrisin], Bactropin, Sulfamethoxazole, Sulfacetamide, Aczone, Cotrimoxazole, Sulfathiazole, Silver Sulfathiazole, Clindamycin, Macrolides, Roxithromycin [Roximycin], Erythrocin, Zmax, Zithromax, Biaxin, Ery-Tab, Dificid, Erythrocin Stearate Filmtab, Biaxin XL, E.E.S. Granules, E.E.S.-400 Filmtab, Eryc, EryPed, Erythrocin Lactobionate, Ilosone, PCE Dispertab, Erythromycin [E-Mycin], Josamycin, [Josalid], Pristinamycin, [Pyostacine], Azithromycin, Clarithromycin, Spiramycin [Spirex], Telithromycin [Ketek], Dirithromycin [Dynabac], Fusidic Acid, Fucidin, Fucithalmic, Stafine, Sodium Fusidate, Fucicort, Fucibet, Fucizon, Fusimax, Foban, Stanicid, Fugen, Optifucin, Taksta, Phudicin, Usidin, Chloramphenicol [Clorin], Thiamphenicol [Biothicol], Metronidazole [Flagyl], Linezolid [Zyvox], Tedizolid [Sivextro], Retapamulin [Altabax], Nitrofurantoin, Trimethoprim, Mupirocin, Gramicidin, and Fosfomycin.

The presently disclosed formulation can include formulations having the mixture of at least two antibiotics comprising two or more antibiotics selected from the group consisting of neomycin sulfate, polymyxin B sulfate, polymyxin E, polymyxin M, bacitracin, streptomycin, kanamycin, tobramycin, gentamicin, and paromomycin.

In further embodiments of the presently disclosed formulation, the vitamin A derivative can be one or more vitamin A derivatives selected from the group consisting of tretinoin, retinaldehyde, retinol, retinyl palmitate, tazarotene, α-retinoic acid, β-retinoic acid, γ-retinoic acid, trans-retinoic acid, 9-cis-retinoic acid, etretinate, acitretin, adapalene, bexarotene, and tazarotene.

Additional embodiments of the presently disclosed formulation can further comprise one of more members selected from the group consisting of aloe vera, methylsulfonylmethane, sodium sulfacetamide, dimethyl sulfoxide, and sulfonamide compounds.

This disclosure also includes a method of wound healing by inhibiting production of at least one of inflammatory cells, neutrophils, macrophages, and leukocyte lineages in a wound area, limiting inflammation of the wound area, and then decreasing degradation of platelets to thereby modulate the coagulation process and formation of a clot by application of a formulation comprised of a steroid, a mixture of at least two antibiotics, and a vitamin A derivative.

The presently taught method includes the additional steps of controlling coagulation lineages to stop conversion of prothrombin into thrombin, and preventing conversion of fibrinogen into fibrin to thereby prevent formation of a primary blood clot.

An additional step of adding antibiotics to the wound area to thereby decrease microbial infections can also be included in the presently disclosed method.

This method can include several additional steps, such as, vasoconstricting blood vessels, and then decreasing the production of bradykinin peptide and histamine by decreasing cytokine secretion. The method can, in some embodiments, further comprise restricting the flow of neutrophils and M1 macrophages into the wound area. The decreased cytokine secretion is believed to be due to reduced levels of NF-κB present in the supplying cell's nucleus.

Some embodiments of the present method can further include limiting migration of leukocytes and platelets to the wound area.

Other embodiments can further include placing and maintaining the wound area in hemostasis thereby reducing the transfer of bacteria to the blood supply. These embodiments can further utilize the steps of activating M2 macrophages, phagocytizing of debris and dead tissue in the wound area by the activated M2 macrophages, proliferating of fibroblasts, and secreting of NF-κB factors that attract and proliferate endothelial cells to promote angiogenesis and new tissue formation.

The teachings of this application also include a method of preparing a pharmaceutical formulation comprising providing a pharmaceutical base formulation, providing pharmaceutical grade 1% hydrocortisone, mixing the base formulation with the hydrocortisone to produce a first mixture, aging the first mixture at room temperature for a first time period, providing at least three different antibiotic-containing formulations, mixing the at least three antibiotic-containing formulations together to produce a second mixture, aging the second mixture at room temperature for a second time period, then adding the second mixture to the first mixture to produce a third mixture, aging the third mixture at room temperature for a third time period, providing a vitamin A derivative-containing formulation, adding the vitamin A derivative-containing formulation to the third mixture to produce a final formulation, and aging the final formulation at room temperature for a fourth time period.

This preparative method can include a first time period of at least seven days, a second time period of at least 14 days, a third time period of between two weeks and two months, and a fourth time period of between seven and 10 days.

The pharmaceutical base formulation utilized in the disclosed preparative method can be comprised any one of glyceryl monostearate, polyoxyl 40 stearate, glycerin, paraffin, stearyl alcohol, isopropyl palmitate, sorbitan mono-stearate, benzyl alcohol, potassium sorbate, lactic acid, and purified water.

This pharmaceutical formulation prepared by this method can include a mixture of at least three antibiotic-containing formulations which comprises three or more antibiotic-containing formulations selected from the group consisting of neomycin sulfate, polymyxin B sulfate, polymyxin E, polymyxin M, bacitracin, streptomycin, kanamycin, tobramycin, gentamicin, paromomycin, Tobrex, Garamycin, Neomycin, Rifampicin, Rifaldazine, Rofact, Rfamycin, Cephalosporins N, C, P, Ancef, Kefazol[cefazolin], Ceclor, Cefazolin Cefaclor, Cefdinir, Ceftin, Zinacef [Cefuroxime], Duricef, Velocef, Biocef, Panixine [cefadroxil], Keflex, Keftabs [cephalexin], Maxipime [cefepime], Rocephin [ceftria], Avibactam/Ceftazidime, Ceftolozane/Tazobactam [Teflaro], Vantin, Omnicef, Cedax, Claforan, Vantin, Spectracef, Tazicef, Fortaz, Ceptaz, Cefobid, Cefizox, Spectracef [SUPRAX], Penicillins, Penicillin V [Phenoxymethylpenecillin], Penicillin G [BenzylPenicillin] [pfizerpen, permapen], Amoxcillin [amoxil], Ampicillin [unasyn] [Polycillin], Carbenecillin, Cloxacillin [Cloxapen], Augmentin [amox/clavulonate], Nafcillin [nallpen], Oxacillin [bactocill], Dicloxacillin [Diclocil], Flucloxacillin [Floxapen], Glycopeptides, anti-infective antibiotics, Vancomycin [for MRSA], Teicoplanin, Telavancin, Ramoplanin, Decaplanin, Bleomycin, Ciprofloxacin, Nalidixic Acid, Norfloxacin, Levofloxacin, Gemifloxacin, Moxifloxacin, Doxycycline, Methacycline, Minocycline, Oxytetracycline, Chlortetracycline, Lymecycline, Co-Trinoxazole [septrin], Sulfadiazine, Sulfamethoxazole [gantanol], Trimethoprim-Sulfamethoxazole [bactrim, bactrimds, septra, septrads], Trimethoprim [trimpex, proloprim, and primsol], Sulfasalazine [azulfidine EN-tabs, Azulfidine, and Sulfazine], Sulfisoxazole [Gantrisin], Bactropin, Sulfamethoxazole, Sulfacetamide, Aczone, Cotrimoxazole, Sulfathiazole, Silver Sulfathiazole, Clindamycin, Macrolides, Roxithromycin [Roximycin], Erythrocin, Zmax, Zithromax, Biaxin, Ery-Tab, Dificid, Erythrocin Stearate Filmtab, Biaxin XL, E.E.S. Granules, E.E.S.-400 Filmtab, Eryc, EryPed, Erythrocin Lactobionate, Ilosone, PCE Dispertab, Erythromycin [E-Mycin], Josamycin, [Josalid], Pristinamycin, [Pyostacine], Azithromycin, Clarithromycin, Spiramycin [Spirex], Telithromycin [Ketek], Dirithromycin [Dynabac], Fusidic Acid, Fucidin, Fucithalmic, Stafine, Sodium Fusidate, Fucicort, Fucibet, Fucizon, Fusimax, Foban, Stanicid, Fugen, Optifucin, Taksta, Phudicin, Usidin, Chloramphenicol [Clorin], Thiamphenicol [Biothicol], Metronidazole [Flagyl], Linezolid [Zyvox], Tedizolid [Sivextro], Retapamulin [Altabax], Nitrofurantoin, Trimethoprim, Mupirocin, Gramicidin, and Fosfomycin.

In some embodiments of the present method, the three different antibiotic-containing formulations are selected from the group consisting of neomycin sulfate, polymyxin B sulfate, polymyxin E, polymyxin M, bacitracin, streptomycin, kanamycin, tobramycin, gentamicin, and paromomycin.

The presently disclose method further comprises mixing periodically the mixtures during each of the respective aging steps.

The vitamin A derivative utilized in the presently disclosed method can include one or more vitamin A derivatives selected from the group consisting of tretinoin, retinaldehyde, retinol, retinyl palmitate, tazarotene, α-retinoic acid, β-retinoic acid, γ-retinoic acid, trans-retinoic acid, 9-cis-retinoic acid, etretinate, acitretin, adapalene, bexarotene, and tazarotene.

The presently disclosed compound can be applied directly into an open wound of nearly any size. A sterile gauze can be coated with the ointment then applied to the wound along with, if necessary, firm pressure until any bleeding has ceased and relief is felt at the wound site.

It is recommended to use a wrap or bandage to hold the fresh gauze firmly in place, and leave on overnight or up to two days. The gauze should be firmly in place so the ointment can be in continuous contact with the dermis tissue, and also to protect the wound from any trauma.

In case hematomas appear, the process can be repeated by applying the presently disclosed ointment to the surface of the gauze, and pressing firmly down until hematomas disappear. One should exercise care to avoid any cross contamination by using sterile gauze.

Applicant's experience with patients treated with the presently disclosed composition has been that due to little to no pain or stiffness associated with the treated wound site is that it is not uncommon for the patient to forget about the wound, thus secondary traumas to the wound site can occur. Consequently, with the presently disclosed composition, it can be imperative that the wound site be protected during the treatment period.

Typically, the initial treatment of an open type wound should be no longer than two days. Skin irritation may start to occur after the second day. Use of an antibiotic cream or a petroleum-based ointment applied lightly to the gauze is recommended if a patient wants to keep the wound site covered. After the third day the wound is generally sealed, should be difficult to reopen, and additionally should not be susceptible to infection.

In the case of wounds that do require sutures to hold separated tissues together, (a wound of first intention) it can take considerably longer for the presently disclosed ointment to have an effect on the wound. In these cases, it is recommended that the ointment cover the entire gauze, which in turn, should cover the entire wound area, and can be held firmly in place by a bandage or wrap. This application method can allow the ointment to occlude through the outer layer of the skin and make contact with the underlying vascular layer, or dermis layer. Since the occlusion occurs slowly, in some instances, the presently disclosed ointment can be applied before surgery and can result in less blood loss and surgical complications.

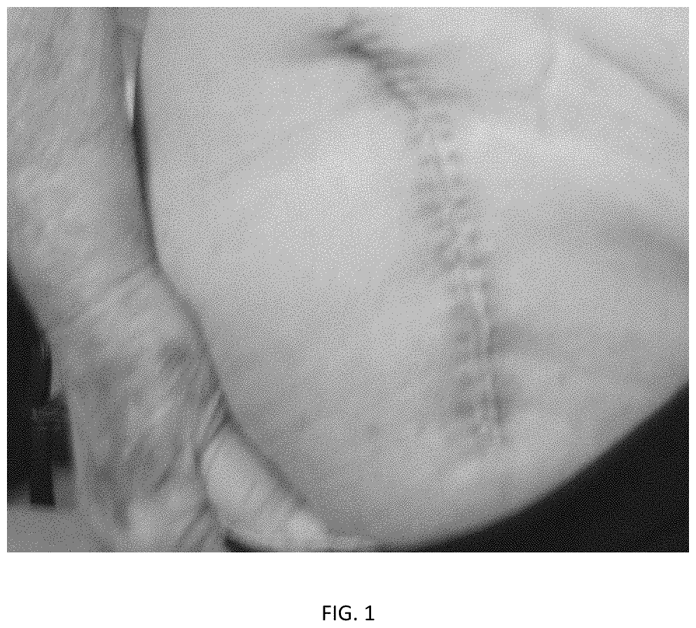

The effectiveness of the presently disclosed formulation and method can be seen in the photographs filed herewith. FIG. 1 shows a 70 year old female patient one week after total hip replacement surgery. The surgical staples are still in place and severe bruising can be seen on her wrist from the intravenous needle insertion while there is a lack of bruising around the staples. The presently disclosed formulation had been applied to the incision area for 20 minutes prior to surgery. The presently disclosed formulation had been generously applied to the incision area soon after surgery, covered with gauze and then removed after 2 days. After seven days, the presently disclosed formulation was reapplied to the incision and covered with gauze for 2 days before being removed.

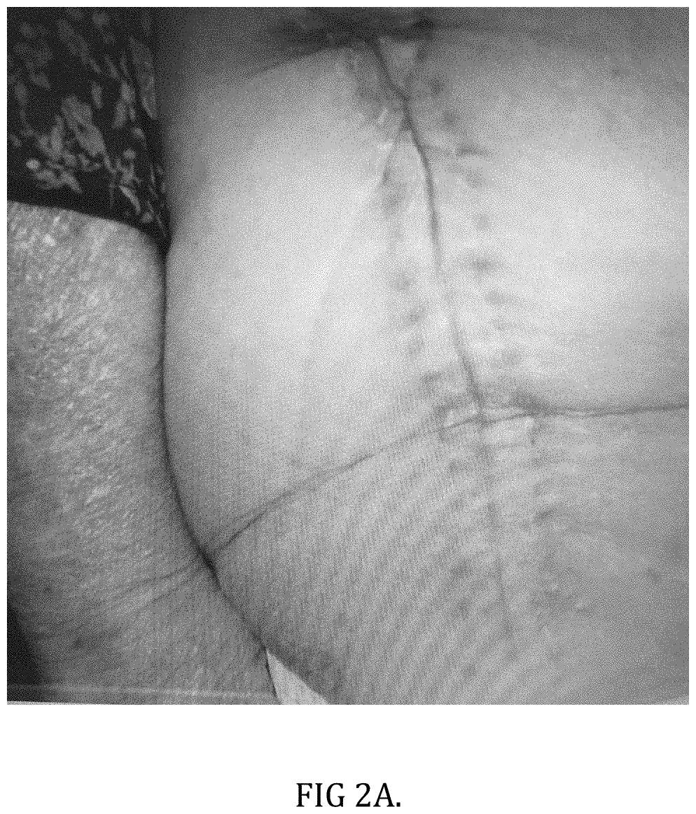

FIG. 2A shows the same female patient seven days after FIG. 1 was taken. The surgical staples have now been removed. It should also be noted that the holes in the skin from where the surgical staples had been inserted are now farther apart than in FIG. 1. It is currently understood that this separation is due to regeneration and growth of new cell tissue, or skin in the incision area from the application of the presently disclosed formulation.

FIG. 2B shows the same female patient fourteen days after FIG. 2A was taken. Notice is taken of the normal healthy coloration of the skin in the incision areas, and the very faint scar from the incision. A definite contrast can be seen with the lack of healing that has occurred on her untreated wrist area. This result is typical wound healing for a surgical incision when treated with the presently disclosed formulation.

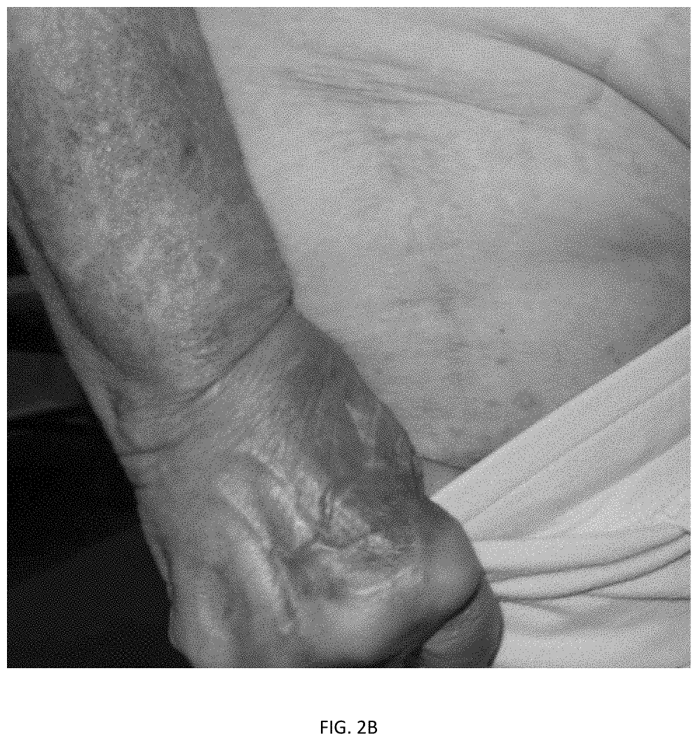

An initial scalp wound is pictured in FIG. 3. This wound was treated with the presently disclosed formulation by applying a generous coating to the wound area and leaving the wound uncovered for two days. Then the presently disclosed formulation was gently removed from the wound area.

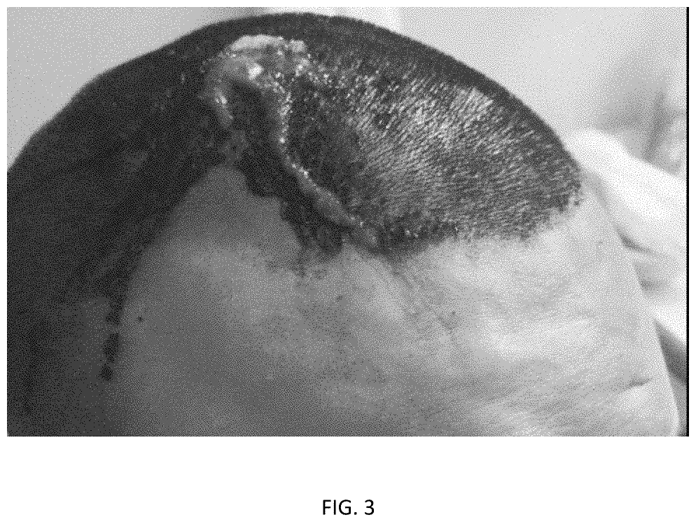

The partially healed scalp wound after three days of treatment is pictured in FIG. 4. The swelling of the wound has decreased significantly, and the wound itself has started to close.

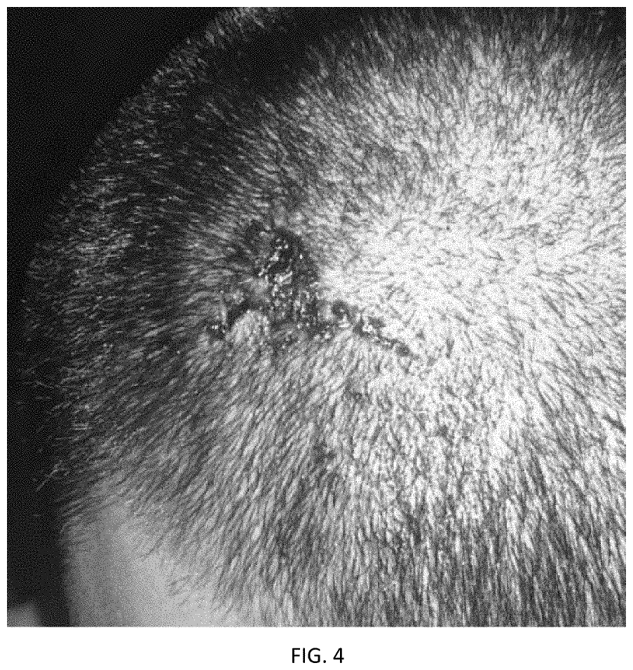

A hand wound from a burn is pictured in FIG. 5. This wound was treated with the presently disclosed formulation by applying a generous coating to the wound area and leaving the wound uncovered for two days. Then the presently disclosed formulation was gently removed from the wound area.

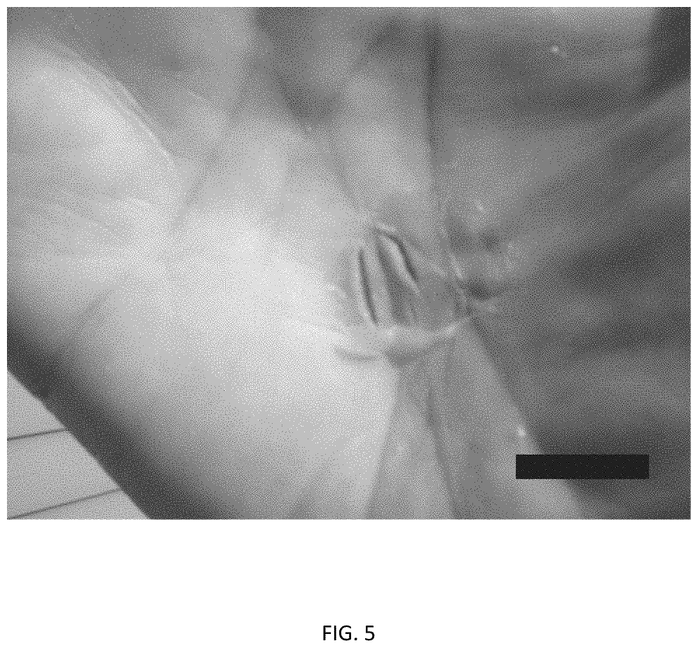

The healed hand burn wound 16 days after the treatment described above is pictured in FIG. 6. Cuts on the hand healed shut without any noticeable scarring.

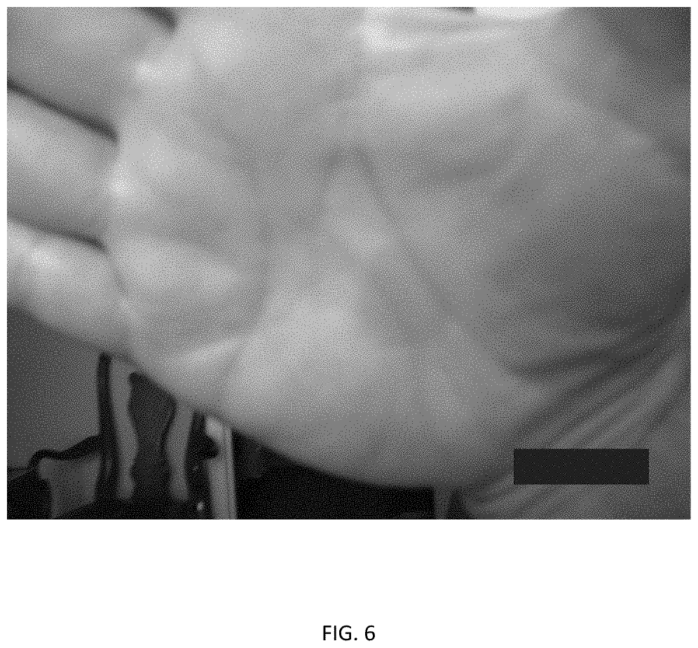

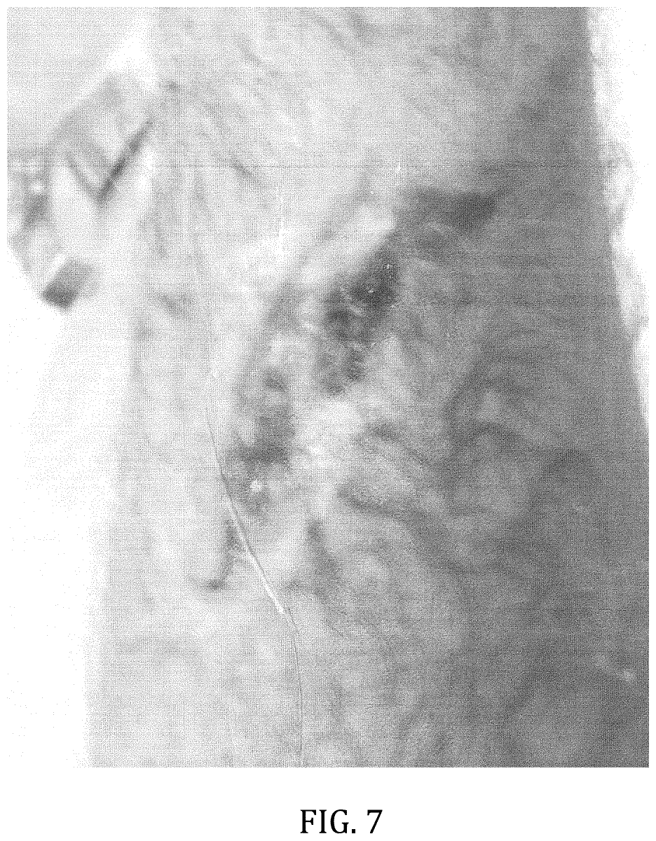

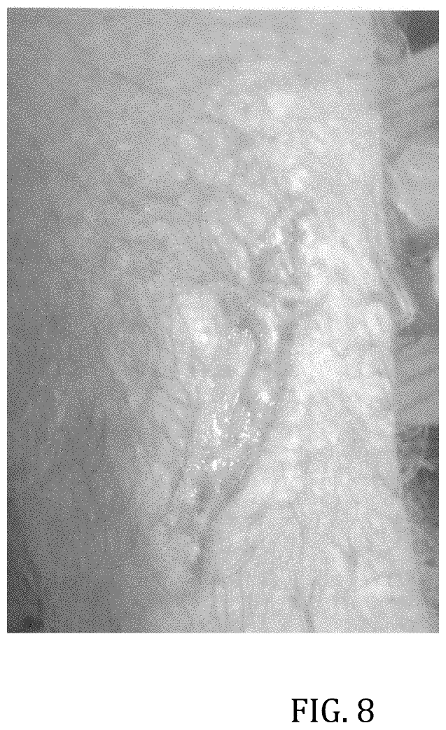

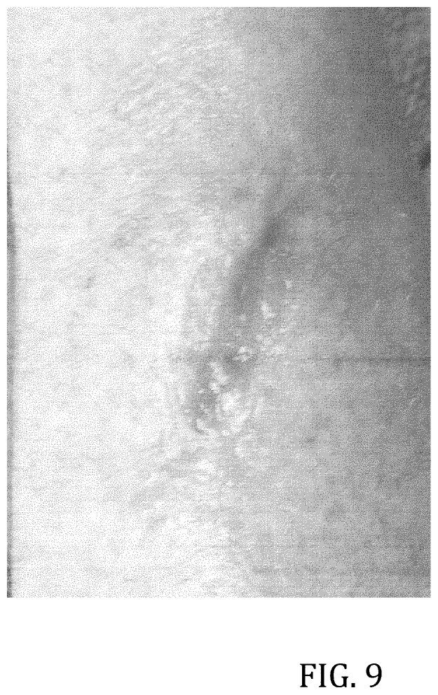

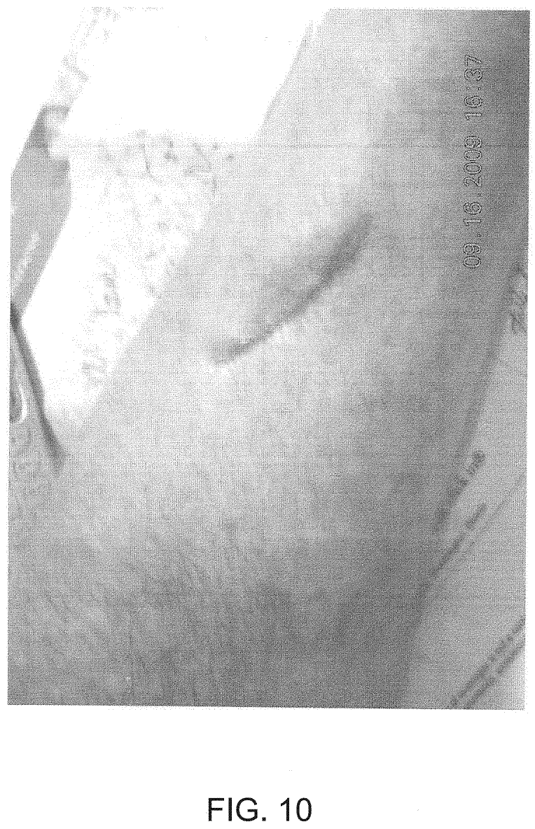

In FIG. 7 is shown a deep laceration on a man's forearm soon after the wound occurred. A liberal coating of the presently disclosed formulation was applied to the wound and covered with a sterile dressing. The dressing was changed every day for 3 days and the presently disclosed formulation was reapplied each day. FIG. 8 shows the same wound 11 days after the first photograph. FIG. 9 shows the same wound after 20 days after the first photograph. FIG. 10 was taken 35 days after the first photograph. The photograph, FIG. 11, shows the laceration completely closed with hair follicles alive in the close vicinity to the laceration.

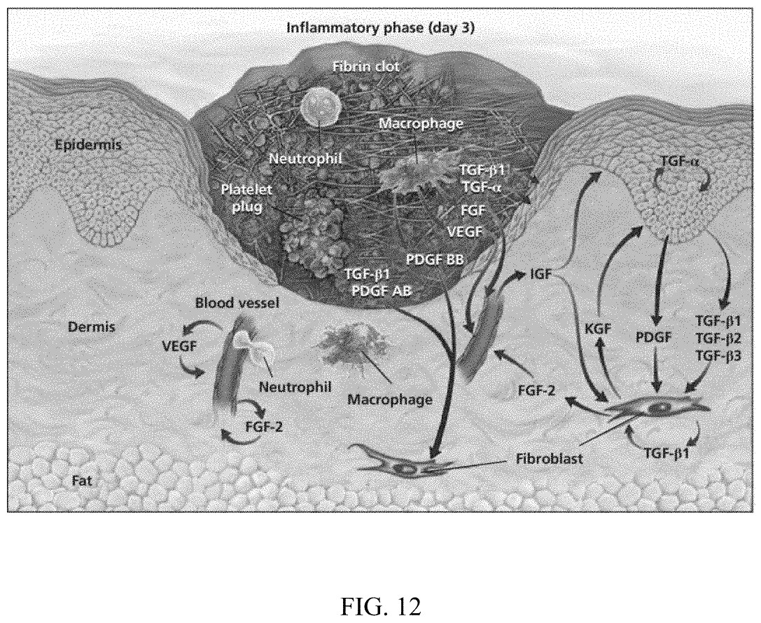

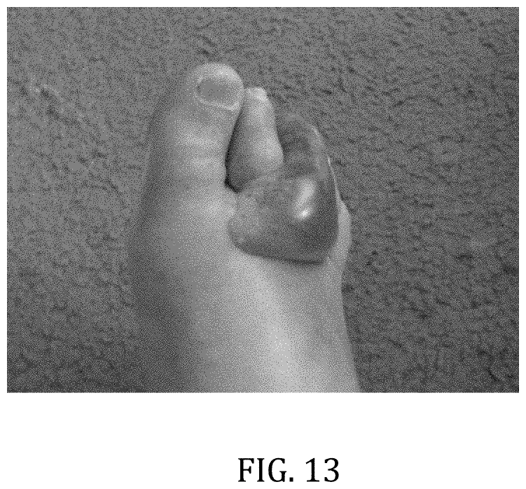

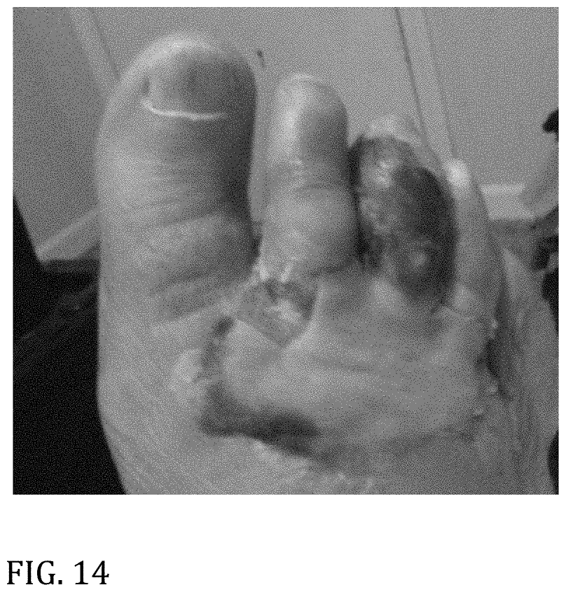

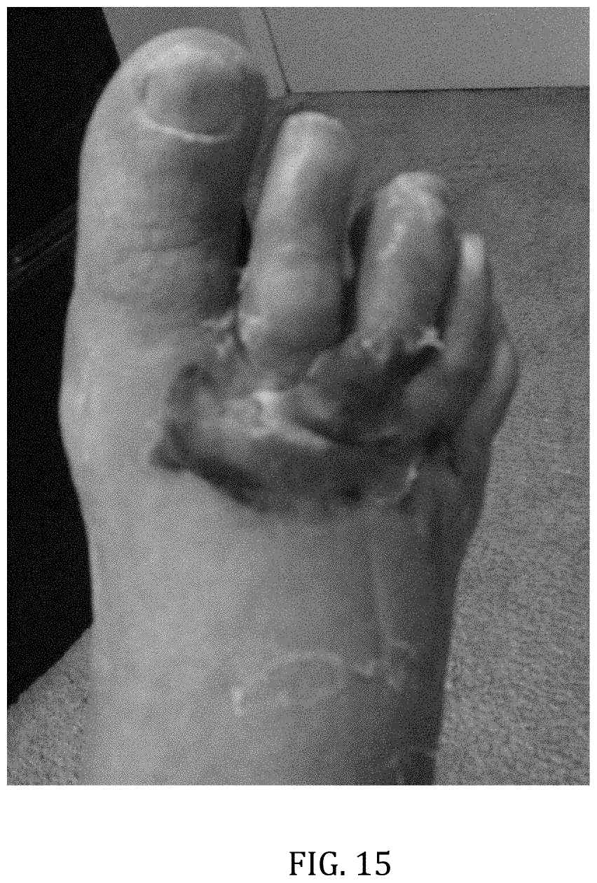

An example of use of the presently disclosed formulation on skin ailments other than cuts is illustrated in FIGS. 13-16, where a reported staph infection on a foot was first treated with conventional means in a hospital, and then with the presently disclosed formulation. FIG. 13 shows the initial infected foot prior to any treatment. FIG. 14 shows the foot after the wound has been drained and the patient received antibiotics by IV for a week; patient reported pain from the foot. FIG. 15 is after five days of topical treatment with the presently disclosed formulation; it is understood that the dead tissue is being phagocytized by macrophages and new tissue is being generated. FIG. 16 shows the foot after about 14 days of continued treatment with the presently disclosed formulation; no more pain is reported by the patient, and no scar is visible to patient at this point.

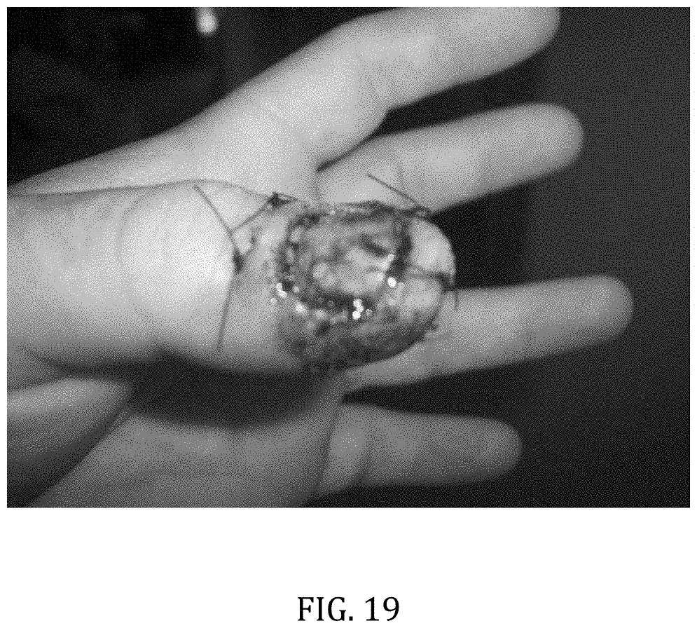

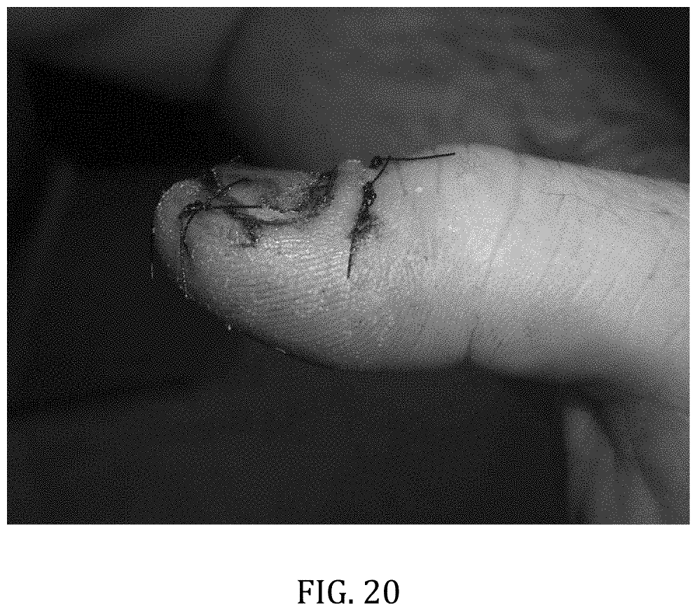

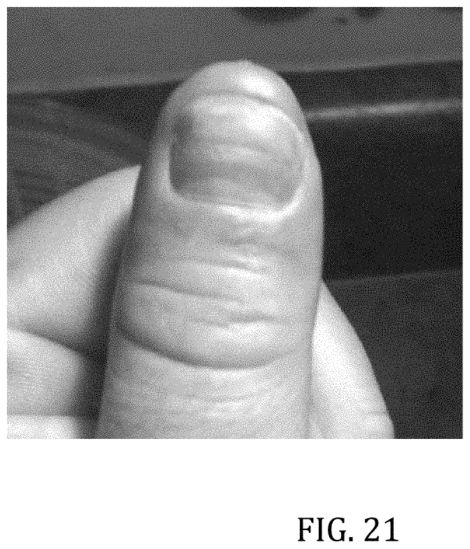

FIG. 19 shows a male patient's thumb which has suffered a very deep accidental cut, and underwent surgery to close the wound. The presently disclosed formulation was then applied to the wound area and wrapped tightly to force the formulation into and past the sutured cut. The dressing was changed every day and new formulation was applied. This procedure was repeated for a total of 14 days, and the result, after seven days is shown in FIG. 20. The healed thumb, without any scabbing or scarring occurring, is shown in FIG. 21, after another seven days of treatment with the presently disclosed formulation.

In general to close a wound of first intention, the ointment can be applied directly inside the wound and then wrapped with a gauze or bandage to push the presently disclosed formulation into the wound, and keep the wound closed. Typically, within 24 hours to 48 hours the sides of the wound will have started to attach securely to one another, and generally after three days, the gauze can be removed and the wound can continue the healing process. The present healing process is believed to occur from the inside of the wound site outwards, and generally without pain or, at least, a lower level of pain than expected.

Usually, an open wound should have an application of the presently disclosed formulation once a day for three days with a wrap or cover applied for at least the first three days. With a wound that has sutures applied, then an application of the presently disclosed formulation once a day for the first two weeks with a tight wrap or cover applied to force the presently disclosed formulation into the sutured opening.

In the case of major surgery such as hip replacement, prior to surgery, the presently disclosed ointment can be applied to the surgical incision area, and covered for twenty minutes before incision. This treatment can result in minimum inflammation of the tissue and minimal bleeding into the surgical area as compared to untreated incisions. The ointment can also be applied into the surgical cavity itself to prevent any further bleeding. Finally, the ointment can be applied to the surgical incision site after closing either directly, or by application to gauze which is left in contact with the incision site at least overnight. This treatment can prevent wound related pain, stiffness, and infection. In some cases, application of the ointment to the surgical incision site can prevent excessive fluid drainage from the incision.

As used herein, “wound” or “wound area” are defined to mean but are not limited to a) lacerations which are irregular tear-like openings in the skin typically caused by blunt trauma; b) incision wounds which are caused by a sharp-edged object such as, for example, a surgeon's knife or glass splinter; c) abrasions which are surface wounds in which a portion of the layers of the epidermis is removed or scraped off; d) puncture wounds which are caused by an object puncturing the skin, and e) burns including first, second and third degree burns.

While “wound healing composition” or variants thereof are used in the present specification to describe the presently disclosed chemical composition, it should be understood that the presently disclosed chemical composition can be used in variety of other situations and is not at all limited to the healing of wounds.

All publications, articles, papers, patents, patent publications, and other references cited herein are hereby incorporated by reference herein in their entireties for all purposes.

Although the foregoing description is directed to the preferred embodiments of the present teachings, it is noted that other variations and modifications will be apparent to those skilled in the art, and which may be made without departing from the spirit or scope of the present teachings.

### EXPERIMENTAL

### Example 1

A pharmaceutical grade 1% hydrocortisone cream comprised of 10 mg of hydrocortisone per gram in a base containing glyceryl monostearate, polyoxyl 40 stearate, glycerin, paraffin, stearyl alcohol, isopropyl palmitate, sorbitan monostearate, benzyl alcohol, potassium sorbate, lactic acid, purified water to reach of a total weight of 16 grams is utilized. This cream mixture is vigorously mixed, covered, and aged at room temperature, and no great than 85 F, for at least seven days. The cream mixture should be stirred for five minutes every other day.

Then, 16 ounces each of three antibiotics, neomycin sulfate (3.5 mg/gram), polymyxin B sulfate (6,000 units/16 ounces), and bacitracin (500 units/16 ounces), are mixed together and allowed to age in sterile conditions for at least two weeks and up to four weeks. Three weeks of aging at room temperature, and no great than 85 F, appear to provide the best results.

The neomycin sulfate is 3.5 mg per gram in a white petroleum base. It is equivalent to 600 μg of neomycin per mg. The polymyxin B sulfate is not less than 6,000 polymyxin units in a white petroleum base. The bacitracin is not less than 500 bacitracin units in a white petroleum base. Each of the antibiotics can be pharmaceutical grade.

After aging, the antibiotic mixture is added to the hydrocortisone cream, and the two mixtures vigorously stirred with the temperature of the mixture not allowed to exceed 85 F (30 C.) This mixture is then held at room temperature for up to two months; with stirring every week. After the aging the mixture should now be an off-white in color with no separation between the hydrocortisone and antibiotic mixtures.

Tretinoin in the form of a 0.1 percent gel is added until ten percent by weight is reached. This mixture is stirred vigorously under sterile conditions until no separation is observed. This mixture is aged for 10 days with stirring every 4 days.

Water is then removed from the mixture by covering the open top of the container with a paper filter, and storing in a clean room at room temperature for a month. The filters are changed weekly.

The above steps should be carried out in a sterile environment with the mixture not being exposed to temperatures above 85 F (30 C) and not exposed to metal surfaces during the aging steps. All ingredients should be at least pharmaceutical grade.

The foregoing detailed description of the various embodiments of the present teachings has been provided for the purposes of illustration and description. It is not intended to be exhaustive or to limit the present teachings to the precise embodiments disclosed. Many modifications and variations will be apparent to practitioners skilled in this art. The embodiments were chosen and described in order to best explain the principles of the present teachings and their practical application, thereby enabling others skilled in the art to understand the present teachings for various embodiments and with various modifications as are suited to the particular use contemplated. It is intended that the scope of the present teachings be defined by the following claims and their equivalents.

What we claim is:

1. A method of promoting tissue regeneration in a mammal comprising:
providing a formulation comprised of a steroid, a mixture of at least two antibiotics, and a vitamin A derivative,
applying the formulation to a wound;
thereby reducing bleeding from any opened blood vessels in the wound by vasoconstriction, and
preventing formation of a clot within the wound.

2. The method according to claim 1, further comprising reducing a flow of one or more members of the group consisting of leukocytes, peptide regulatory factors, glycoproteins, cytokines, membrane cytokines, soluble cytokines, growth factors, TGF-b, PDGF, platelets, TNF-a, IL8, chemokines, histamine, bradykinin, neutrophils, M1 macrophages, fibroblasts, keratinocytes, plasmin, prothrombin, prostaglandins, kinins, liposomal enzymes, and thrombin into the wound.

3. The method according to claim 2, whereby the absence of one or more members of the group consisting of leukocytes, prostaglandins, kinins, histamine, and liposomal enzymes decreases vascular permeability and inflammation in the wound.

4. The method according to claim 1, further comprising reducing a flow of histamine into the wound.

5. The method according to claim 4, whereby the absence of histamine decreases vascular dilation in the wound area.

6. The method according to claim 1, further comprising reducing the flow of one or more members of the group consisting of histamine, bradykinin, degranulating platelets, and chemokines to prevent the formation of a fibrin clot.

7. The method according to claim 6, whereby the absence of histamine and bradykinin decreases both vascular permeability and dilation in the wound area.

8. The method according to claim 6, whereby the absence of histamine and bradykinin reduces pain in the wound area for the entire duration of the healing process.

9. The method according to claim 1, further comprising stopping the formation of one of more of prothrombin, thrombin, and prothrombinase complex.

10. The method according to claim 1, further comprising reducing the conversion of fibrinogen to fibrin.

11. The method according to claim 1, wherein the formulation is applied directly into the wound.

12. A formulation for wound healing comprising
at least one anti-inflammatory steroid, a mixture of at least two antibiotics, and a vitamin A derivative.

13. The formulation according to claim 12, wherein the steroid comprises one or more steroid selected from the group consisting of hydrocortisone, hydrocortisone valerate, hydrocortisone butyrate, desonide, amcinonide, betamethasone valerate, clobetasone butyrate, desoximetasone, diflucortolone valerate, fluocinolone acetonide, mometasone furoate, prednicarbate, triamcinolone acetonide, flurandrenolide, fluticasone propionate, betamethasone dipropionate, halcinonide, triamcinolone acetonide, clobetasol propionate, halobetasol propionate, temovate, dexamethasone, diflucortolene, flumethasone, fluocinonide, ethylprednisolone, budesonide, alclometasone dipropionate, methylprednisone, and prednisone.

14. The formulation according to claim 12, wherein the mixture of at least two antibiotics comprises two or more antibiotics selected from the group consisting of neomycin sulfate, polymyxin B sulfate, polymyxin E, polymyxin M, bacitracin, streptomycin, kanamycin, tobramycin, gentamicin, paromomycin, Tobrex, Garamycin, Neomycin, Rifampicin, Rifaldazine, Rofact, Rfamycin, Cephalosporins N, C, P, Ancef, Kefazol[cefazolin], Ceclor, Cefazolin Cefaclor, Cefdinir, Ceftin, Zinacef [Cefuroxime], Duricef, Velocef, Biocef, Panixine [cefadroxil], Keflex, Keftabs [cephalexin], Maxipime [cefepime], Rocephin [ceftria], Avibactam/Ceftazidime, Ceftolozane/Tazobactam [Teflaro], Vantin, Omnicef, Cedax, Claforan, Vantin, Spectracef, Tazicef, Fortaz, Ceptaz, Cefobid, Cefizox, Spectracef [SUPRAX], Penicillins, Penicillin V [Phenoxymethylpenecillin], Penicillin G [BenzylPenicillin] [pfizerpen, permapen], Amoxcillin [amoxil], Ampicillin [unasyn] [Polycillin], Carbenecillin, Cloxacillin [Cloxapen], Augmentin [amox/clavulonate], Nafcillin [nallpen], Oxacillin [bactocill], Dicloxacillin [Diclocil], Flucloxacillin [Floxapen], Glycopeptides, anti-infective antibiotics, Vancomycin, [for MRSA], Teicoplanin, Telavancin, Ramoplanin, Decaplanin, Bleomycin, Ciprofloxacin, Nalidixic Acid, Norfloxacin, Levofloxacin, Gemifloxacin, Moxifloxacin, Doxycycline, Methacycline, Minocycline, Oxytetracycline, Chlortetracycline, Lymecycline, Co-Trinoxazole [septrin], Sulfadiazine, Sulfamethoxazole [gantanol], Trimethoprim-Sulfamethoxazole [bactrim, bactrimds, septra, septrads], Trimethoprim [trimpex, proloprim, and primsol], Sulfasalazine [azulfidine EN-tabs, Azulfidine, and Sulfazine], Sulfisoxazole [Gantrisin], Bactropin, Sulfamethoxazole, Sulfacetamide, Aczone, Cotrimoxazole, Sulfathiazole, Silver Sulfathiazole, Clindamycin, Macrolides, Roxithromycin [Roximycin], Erythrocin, Zmax, Zithromax, Biaxin, Ery-Tab, Dificid, Erythrocin Stearate Filmtab, Biaxin XL, E.E.S. Granules, E.E.S.-400 Filmtab, Eryc, EryPed, Erythrocin Lactobionate, Ilosone, PCE Dispertab, Erythromycin [E-Mycin], Josamycin, [Josalid], Pristinamycin, [Pyostacine], Azithromycin, Clarithromycin, Spiramycin [Spirex], Telithromycin [Ketek], Dirithromycin [Dynabac], Fusidic Acid, Fucidin, Fucithalmic, Stafine, Sodium Fusidate, Fucicort, Fucibet, Fucizon, Fusimax, Foban, Stanicid, Fugen, Optifucin, Taksta, Phudicin, Usidin, Chloramphenicol [Clorin], Thiamphenicol [Biothicol], Metronidazole [Flagyl], Linezolid [Zyvox], Tedizolid [Sivextro], Retapamulin [Altabax], Nitrofurantoin, Trimethoprim, Mupirocin, Gramicidin, and Fosfomycin.

15. The formulation according to claim 14, wherein the mixture of at least two antibiotics comprises two or more antibiotics selected from the group consisting of neomycin sulfate, polymyxin B sulfate, polymyxin E, polymyxin M, bacitracin, streptomycin, kanamycin, tobramycin, gentamicin, and paromomycin.

16. The formulation according to claim 12, wherein the vitamin A derivative comprises one or more vitamin A derivative selected from the group consisting of tretinoin, retinaldehyde, retinol, retinyl palmitate, tazarotene, α-retinoic acid, β-retinoic acid, γ-retinoic acid, trans-retinoic acid, 9-cis-retinoic acid, etretinate, acitretin, adapalene, bexarotene, and tazarotene.

17. The formulation according to claim 12, wherein formulation further comprises one of more member selected from the group consisting of aloe vera, methylsulfonylmethane, sodium sulfacetamide, dimethyl sulfoxide, and sulfonamide compounds.

18. A method of wound healing comprising
inhibiting production of at least one of inflammatory cells, neutrophils, macrophages, and leukocyte lineages in a wound area,
limiting inflammation of the wound area, and
decreasing degradation of platelets;
thereby modulating the coagulation process and formation of a clot by application of a formulation comprised of a steroid, a mixture of at least two antibiotics, and a vitamin A derivative.

19. The method according to claim 18, further comprising
controlling coagulation lineages to stop conversion of prothrombin into thrombin,
preventing conversion of fibrinogen into fibrin, and
thereby preventing formation of a primary blood clot.

20. The method according to claim 18, further comprising adding antibiotics to the wound area to thereby decrease microbial infections.

21. The method according to claim 18, further comprising
vasoconstricting blood vessels.

22. The method according to claim 21, further comprising
decreasing the production of bradykinin peptide and histamine by decreasing cytokine secretion,
wherein the decreased cytokine secretion is due to reduced levels of NF-κB present in the supplying cell's nucleus.

23. The method according to claim 21, further comprising
limiting migration of leukocytes and platelets to the wound area.

24. The method according to claim 18, further comprising
placing and maintaining the wound area in hemostasis thereby reducing the transfer of bacteria to the blood supply.

25. The method according to claim 24, further comprising
activating M2 macrophages,
phagocytizing of debris and dead tissue in the wound area by the activated M2 macrophages,
proliferating of fibroblasts, and
secreting of NF-κB factors that attract and proliferate endothelial cells to promote angiogenesis and new tissue formation.

26. The method according to claim 21, further comprising
restricting the flow of neutrophils and M1 macrophages to the wound area.

27. A method of preparing a pharmaceutical formulation comprising
providing a pharmaceutical base formulation,
providing pharmaceutical grade 1% hydrocortisone,
mixing the base formulation with the hydrocortisone to produce a first mixture,
aging the first mixture at room temperature for a first time period,
providing at least three different antibiotic-containing formulations,
mixing the at least three antibiotic-containing formulations together to produce a second mixture,
aging the second mixture at room temperature for a second time period,
then adding the second mixture to the first mixture to produce a third mixture,
aging the third mixture at room temperature for a third time period,
providing a vitamin A derivative-containing formulation,
adding the vitamin A derivative-containing formulation to the third mixture to produce a final formulation, and
aging the final formulation at room temperature for a fourth time period.

28. The method according to claim 27, wherein the first time period is at least seven days, the second time period is at least 14 days, the third time period is between two weeks and two months, and the fourth time period is between seven and 10 days.

29. The method according to claim 27, wherein the pharmaceutical base formulation is comprised of glyceryl monostearate, polyoxyl 40 stearate, glycerin, paraffin, stearyl alcohol, isopropyl palmitate, sorbitan mono-stearate, benzyl alcohol, potassium sorbate, lactic acid, and purified water.

30. The method according to claim 27, wherein the mixture of at least three antibiotic-containing formulations comprises three or more antibiotic-containing formulations selected from the group consisting of neomycin sulfate, polymyxin B sulfate, polymyxin E, polymyxin M, bacitracin, streptomycin, kanamycin, tobramycin, gentamicin, paromomycin, Tobrex, Garamycin, Neomycin, Rifampicin, Rifaldazine, Rofact, Rfamycin, Cephalosporins N, C, P, Ancef, Kefazol[cefazolin], Ceclor, Cefazolin Cefaclor, Cefdinir, Ceftin, Zinacef [Cefuroxime], Duricef, Velocef, Biocef, Panixine [cefadroxil], Keflex, Keftabs [cephalexin], Maxipime [cefepime], Rocephin [ceftria], Avibactam/Ceftazidime, Ceftolozane/Tazobactam [Teflaro], Vantin, Omnicef, Cedax, Claforan, Vantin, Spectracef, Tazicef, Fortaz, Ceptaz, Cefobid, Cefizox, Spectracef [SUPRAX], Penicillins, Penicillin V [Phenoxymethylpenecillin], Penicillin G [BenzylPenicillin] [pfizerpen, permapen], Amoxcillin [amoxil], Ampicillin [unasyn] [Polycillin], Carbenecillin, Cloxacillin [Cloxapen], Augmentin [amox/clavulonate], Nafcillin [nallpen], Oxacillin [bactocill], Dicloxacillin [Diclocil], Flucloxacillin [Floxapen], Glycopeptides, anti-infective antibiotics, Vancomycin [for MRSA], Teicoplanin, Telavancin, Ramoplanin, Decaplanin, Bleomycin, Ciprofloxacin, Nalidixic Acid, Norfloxacin, Levofloxacin, Gemifloxacin, Moxifloxacin, Doxycycline, Methacycline, Minocycline, Oxytetracycline, Chlortetracycline, Lymecycline, Co-Trinoxazole [septrin], Sulfadiazine, Sulfamethoxazole [gantanol], Trimethoprim-Sulfamethoxazole [bactrim, bactrimds, septra, septrads], Trimethoprim [trimpex, proloprim, and primsol], Sulfasalazine [azulfidine EN-tabs, Azulfidine, and Sulfazine], Sulfisoxazole [Gantrisin], Bactropin, Sulfamethoxazole, Sulfacetamide, Aczone, Cotrimoxazole, Sulfathiazole, Silver Sulfathiazole, Clindamycin, Macrolides, Roxithromycin [Roximycin], Erythrocin, Zmax, Zithromax, Biaxin, Ery-Tab, Dificid, Erythrocin Stearate Filmtab, Biaxin XL, E.E.S. Granules, E.E.S.-400 Filmtab, Eryc, EryPed, Erythrocin Lactobionate, Ilosone, PCE Dispertab, Erythromycin [E-Mycin], Josamycin, [Josalid], Pristinamycin, [Pyostacine], Azithromycin, Clarithromycin, Spiramycin [Spirex], Telithromycin [Ketek], Dirithromycin [Dynabac], Fusidic Acid, Fucidin, Fucithalmic, Stafine, Sodium Fusidate, Fucicort, Fucibet, Fucizon, Fusimax, Foban, Stanicid, Fugen, Optifucin, Taksta, Phudicin, Usidin, Chloramphenicol [Clorin], Thiamphenicol [Biothicol], Metronidazole [Flagyl], Linezolid [Zyvox], Tedizolid [Sivextro], Retapamulin [Altabax], Nitrofurantoin, Trimethoprim, Mupirocin, Gramicidin, and Fosfomycin.

31. The method according to claim 27, wherein the three different antibiotic-containing formulations are selected from the group consisting of neomycin sulfate, polymyxin B sulfate, polymyxin E, polymyxin M, bacitracin, streptomycin, kanamycin, tobramycin, gentamicin, and paromomycin.

32. The method according to claim 27, further comprising mixing periodically the mixtures during each of the respective aging steps.

33. The method according to claim 27, wherein the vitamin A derivative comprises one or more vitamin A derivatives selected from the group consisting of tretinoin, retinaldehyde, retinol, retinyl palmitate, tazarotene, α-retinoic acid, β-retinoic acid, γ-retinoic acid, trans-retinoic acid, 9-cis-retinoic acid, etretinate, acitretin, adapalene, bexarotene, and tazarotene.
